(19) United States
(12) Patent Application Publication
(10) Pub. No.: US 2004/0210309 A1
(43) Pub. Date: Oct. 21, 2004

(54) Osteophilic implants

(76) Inventors: Alain J Denzer, Basel (CH); James P Simpson, Basel (CH)

Correspondence Address: FINNEGAN, HENDERSON, FARABOW, GARRETT & DUNNER, LLP, 1300 I STREET, NW, WASHINGTON, DC, 20005, US

(21) Appl. No.: 10/492,131

(22) PCT Filed: Oct. 11, 2002
(86) PCT No.: PCT/CH02/00561

(30) Foreign Application Priority Data
Oct. 11, 2001 (CH) 1873/01

(51) Int. Cl. A61F 2/28

(52) U.S. Cl. 623/16.11

(57) ABSTRACT

Osteophilic implant with a roughened hydroxilated and hydrophilic surface, made from titanium or a titanium alloy and suitable for implantation in bones, whereby the implant is characterised in that said implant is treated in the hydroxilated state with high-energy ultra-violet radiation.

The present invention relates to osteophilic implants which are used for insertion into bone and which have considerably improved osteointegration properties, and to processes for their production.

Implants which are used for insertion into bone, for example hip-joint or knee-joint prostheses or pins to be screwed into the jaw for attachment of artificial teeth, are known per se. Such implants preferably consist of titanium or titanium-based alloys, for example titanium/zircon alloys, it being possible for the latter additionally to contain niobium, tantalum or other tissue-compatible metallic additives. A central property of such implants is their osteointegration time, that is to say the time that passes before the bone substance has become connected with sufficient strength and permanently to the implant surface, that is to say has become integrated with it.

The firmness of the anchoring of the implant in the bone can be established by mechanical measurements, namely by measuring the force, whether pulling, pushing, shearing or torque, needed to extract or unscrew the implant anchored in the bone from its anchoring, i.e. bring about a break of the adhesion between the surface of the implant and the bone substance connected to it. Such measurement methods are known per se and are described, for example, in Brunski, Clinical Materials, Vol. 10, 1992, pp. 153-201. Measurements have shown that titanium implants with a smooth surface structure anchor only insufficiently in the bone, whereas implants with a roughened surface afford a distinctly improved bone-implant connection in terms of tensile strength.

EP 0 388 576 therefore proposes applying a macro-roughness to the implant surface, in a first step, by means of sandblasting, and subsequently superimposing a micro-roughness on said macro-roughness by means of treatment in an acid bath. The implant surface is roughened by means of sandblasting, for example, and subsequently treated with an etching agent, e.g. hydrofluoric acid or a hydrochloric acid/sulfuric acid mixture. The surface provided with a defined roughness in this way is then washed with water and solvents and subjected to a sterilizing treatment.

The chemical state of surfaces, for example made of titanium or titanium-based alloys, is complex. It is assumed that the surface of titanium metal spontaneously oxidizes in air and water and that a reaction with water then takes place on the surface, that is to say in the outermost atomic layer, with formation of hydroxyl groups. This surface containing hydroxyl groups is referred to in the literature as a “hydroxylated” surface. See H. P. Boehm, Acidic and Basic Properties of Hydroxylated Metal Oxide Surfaces, Discussions Faraday Society, Vol. 52, 1971, pages 264-275. However, a hydroxylated surface can exert its full effect only when it is in the “hydrophilic” state, that is to say when it is not covered with readily volatile hydrocarbons and other compounds, for example sulfur dioxide or nitrogen monoxide, which are present in the air. At each stage of the production process, other impurities can also end up on the implant surface, for example in the cleaning process if the implant is treated with organic solvents such as methanol or acetone. A hydroxylated and hydrophilic surface, e.g. of titanium or a titanium alloy, has biologically active properties and can be designated as bioactive. A hydroxylated and hydrophilic implant surface fuses with the bone substance and forms a strong union much more quickly than does a similar hydroxylated but non-hydrophlic surface. The production of a hydroxylated and at the same time hydrophilic implant surface is described in WO 0044305 (PCT/EP0/00619).

A hydroxylated surface free of contamination has a high surface energy, which means that readily volatile hydrocarbons and other impurities present in unpurified air are rapidly adsorbed. Initially, the adsorbed hydrocarbons, for example, form a monomolecular layer (monolayer) on the hydroxylated surface, as a result of which the hydrophilic character of the surface is lost. The longer the implant is exposed to the air, however, the thicker the contamination layer becomes. A clean hydroxylated surface made of titanium is hydrophilic and, when wetted with water, has a contact angle of less than 50°, whereas a contaminated hydroxylated surface has a contact angle of over 70° and is designated as hydrophobic. The hydroxylated and hydrophilic surface, and thus its biological activity too, can of course be maintained substantially unchanged by enclosing this surface in an envelope so as to avoid contact between the surface and compounds which may adversely affect the hydrophilic character of the implant surface. However, when the envelope is broken, as is necessary just before implantation, it is not possible to prevent contact between the implant surface and the atmospheric outside air and thus a deterioration in the hydrophilic character of the implant surface. There is therefore a need to produce or treat the hydroxylated and hydrophilic implant surface in such a way that its hydrophilic character is substantially maintained over a lengthy period of time, even upon contact with the atmospheric air.

It has now been found that a hydroxylated but contaminated implant surface, i.e. one covered with impurities and thus rendered hydrophobic, acquires a hydrophilic character when said surface is treated with high-energy ultraviolet radiation (UV rays). The hydroxylated and hydrophilic surface obtained in this way is, surprisingly, much less sensitive to coverage by foreign substances from the atmospheric outside air than is a hydroxylated and hydrophilic implant surface which, for example, has been obtained by acid etching. It has likewise been found that the stability of the hydrophilic character of a hydroxylated and hydrophilic implant surface obtained by acid etching greatly increases if this surface is treated in the hydrophilic state with high-energy ultraviolet radiation. A further advantage of this treatment is that it is possible to dispense with additional sterilization of the implants.

The present invention is defined in the patent claims. In particular, the present invention relates to an osteophilic implant with an especially roughened, hydroxylated and hydrophilic surface, said implant preferably being made of titanium or a titanium alloy and being suitable for implantation in bone, characterized in that the implant was treated with high-energy ultraviolet radiation in the hydroxylated state. Other materials are also conceivable, however, such as other metals, and ceramics.

The expression “implant in the hydroxylated state” designates an implant which was produced with a hydroxylated surface but whose surface has a hydrophobic character, and it also designates an implant with a hydroxylated and hydrophilic surface. The implant according to the invention has a greatly increased stability against loss of the hydrophilic character and thus has improved osteointegration properties.

The present invention also relates to processes for producing an osteophilic implant with an especially roughened, hydroxylated and hydrophilic surface, said implant preferably being made of titanium or a titanium alloy and being suitable for implantation in bone, characterized in that the implant is treated with high-energy ultraviolet radiation in the hydroxylated state.

The implants according to the invention preferably consist of a titanium alloy, preferably of a titanium/zircon alloy, it being possible for the latter additionally to contain niobium, tantalum or other tissue-compatible metallic additives. Other suitable materials can likewise be used within the context of the invention. These implants are preferably used as hip-joint or knee-joint prostheses or as pins to be screwed into the jaw for attachment of artificial teeth. Implants of this type, their characteristics and the metallic materials used to produce them are known per se and are described, for example, in J. Black, G. Hastings, Handbook of Biomaterials Properties, pages 135-200, published by Chapman & Hall, London, 1998.

The structural and functional anchoring, for example of a tooth implant, in the bone is generally achieved by applying a macro-roughness such as a screw thread or depressions in the surface and/or possibly an additional micro-roughness, said micro-roughness being applied either in an additive process by means of plasma technology, or in a subtractive process by chemical etching on the surface. The application of a macro-roughness, such as a screw thread or depressions, in the surface and, if appropriate, an additional micro-roughness is known per se. For the implementation of the present invention, that is to say for production of an implant provided with a macro-roughness on which a micro-roughness is also superposed, the procedure followed is preferaby as described in EP 0 388 576 or in WO 0044305 (PCT/EP00/00619). According to the invention, the implant obtained in this way can then be treated with high-energy ultraviolet radiation.

The implant surface according to the invention preferably has a macro-roughness in the range of 5 μm to 100 μm (in each case peak to valley), in particular in the range of 20 μm to 40 μu, and a superposed micro-roughness in the range of 0.5 μm to 20 μm, in particular in the range of 0.5 μm to 10 μm, in particular about 2 μm.

These two superposed roughnesses can be produced in two consecutive steps. For example, the macro-roughness of the implant surface is generated by sandblasting with corundum particles having an average particle size in the range of 0.25-0.5 mm, after which the micro-roughness is obtained by treatment with an aqueous hydrochloric acid/sulphuric acid mixture with an HCl:H₂SO₄:H₂O ratio of 2:1:1 at a temperature in the range of 80° C. to about 110° C. for about five minutes. After the acid bath, the surface-structured titanium parts, or the implants, are washed thoroughly with pure water to neutral, treated, if appropriate, with alcohol, acetone or another organic solvent or a disinfectant, and dried in hot air. The implants obtained in this way can finally be packaged and, if appropriate, sterilized by gamma radiation. In this process, a hydrophilic implant surface is obtained if, after the acid bath, the implant surface is washed thoroughly with pure water to neutral and dried in an atmosphere inert to the hydrophilic surface, and the implant is stored in an inert atmopsphere. In the case of treatment with alcohol, acetone or another organic solvent or a disinfectant and drying in hot atmospheric air, a hydrophobic implant surface is obtained.

The “pure” water used for washing is preferably water which has been distilled several times or prepared by inverse osmosis and which has preferably been prepared in an inert atmosphere, that is to say, for example, under reduced pressure, in a nitrogen or noble gas atmosphere. Moreover, the pure water has an electrical resistance of at least 2 Mohm cm (electrical resistance >2 Mohm cm) and a total organic carbon (TOC) content of at most 10 ppb (≦10 ppb).

According to the invention, the implant is treated with high-energy ultraviolet radiation in the hydroxylated state. The effect of the high-energy ultraviolet radiation (UV radiation) is that the implant surface becomes hydrophilic and the impurities present on the surface are removed. According to the invention, the high-energy UV radiation employed involves UV rays with a wavelength in the range of 150 nm to 300 nm, preferably in the range of 170 nm to 260 nm, and in particular UV rays in the range of the absorption maximum of oxygen, that is to say with a wavelength of about 184.9 nm, and in the range of the absorption maximum of ozone, that is to say with a wavelength of about 253.7 nm. Wavelengths in this range promote the simultaneous formation and decomposition of ozone and are able to break up high-energy chemical bonds, for example the ethylenic carbon-carbon double bond. Wavelengths in the ranges stated are generated by so-called xenon emitters, which are available on the market.

Since the intensity of the radiation decreases exponentially with the distance from the UV source, it is advantageous that the distance between the implant surface to be treated and the UV source is kept small, for example in the range of about 1 mm. The duration of the UV treatment depends on the nature and thickness of the contamination layer and can be readily established and optimized by the person skilled in the art. The duration of their radiation is generally in the range of 1 minute to 15 minutes. In the treatment, according to the invention, of a hydrophilic (and thus already relatively clean) surface, a cleaner product is also generally obtained than in the case of treatment of a contaminated surface.

For analysis of the implant surfaces treated by UV rays, it is advantageous to use XPS (X-ray-excited photoelectron spectroscopy) or Auger electron spectroscopy (AES). An implant surface, for example as it appears directly after the acid etching and is hydroxylated and hydrophilic, has a wetting angle with water of less than 50° (<50°) upon advance of the water drop in contact with the surface, or of less than 20° (<20°) in the case of the drop reforming, and has an appreciable biological activity. The same applies to an implant surface which is treated with UV rays but whose hydrophilic character, according to the invention, is maintained over a lengthy period of time in contact with the atmospheric outside air.

Industrially produced surfaces of titanium and titanium-based alloys for processing in laboratories and clinics usually have impurities which consist essentially of carbon compounds and traces of nitrogen, calcium, sulphur, phosphorus and silicon. These impurities are concentrated in the outermost metal oxide layer. The hydroxylated and hydrophilic implant surface obtained according to the invention preferably contains not more than 20 atom-% carbon measured by spectroscopic methods such as XPS or AES or other spectroscopic methods known per se.

Subsequent to the UV irradiation, the resulting implant is preferably stored in an envelope filled with gases which are inert to the implant surface. Such inert gases, that is to say gases which do not affect the hydrophilic character of the surface or its biological activity, are for example nitrogen, oxygen or noble gas, such as argon. The envelope is preferably impermeable to gases and liquids. Examples of compounds which can impair the biological activity of the implant surface are, as already mentioned, methanol, ethanol, acetone and related ketones, and numerous other organic compounds, or carbon dioxide.

If appropriate, the envelope can be filled partially or completely with pure water, which can optionally contain additives, this water being present at least in such an amount that moistening or wetting of the roughened implant surface is ensured.

Examples of suitable additives are monovalent alkali metal cations such as Na⁺ or K⁺, with corresponding anions in the form of inorganic salts, for example sodium chloride, potassium chloride, sodium or potassium chlorate, sodium or potassium nitrate, sodium or potassium phosphate, or a mixture of such salts. It is likewise also possible to add divalent cations with the form of water-soluble inorganic salts. Suitable cations are, in particular, Mg⁺², Ca⁺², Sr⁺² and/or Mn⁺² in the form of the chlorides, or mixtures thereof. Suitable anions are also phosphate and phosphonate anions, by which are meant in each case also monoorthophosphate anions and diorthophosphate anions, and monoorthophosphonate anions and diorthophosphonate anions, in combination with the cations mentioned.

Preferred cations and anions are those which already occur in body fluid, especially in the respective physiological concentration and with a physiological acid value (pH) in the range of preferably 4 to 9 and preferably with an acid value in the range of 6 to 8. Preferred cations are Na⁺, K⁺, Mg⁺² and Ca⁺². The preferred anion is Cl⁻. The total amount of said cations and anions preferably in each case lies in the range of about 50 mEq/l to 250 mEq/l, preferably about 100 mEq/l to 200 mEq/l and is preferably about 150 mEq/l. Here, Eq/l means (formula) equivalent weight, and Eq/l corresponds to the atomic weight of the formula unit divided by the valency. mEq/l means milliequivalent weight per liter. If the envelope contains divalent cations, in particular Mg⁺², Ca⁺², Sr⁺² and/or Mn⁺², alone or in combination with the monovalent cations mentioned, then the total amount of the divalent cations present is preferably in the range of 1 mEq/l to 20 mEq/l.

The gas-tight and liquid-tight envelope is preferably a sealed ampule made of glass, metal, a synthetic polymer or another gas-tight and liquid-tight material, or consists of a combination of these materials. The metal is preferably in the form of a thin metal sheet, it being possible to combine polymeric materials and metallic sheets, but also glass, in a manner known per se with one another to give a suitable packaging.

However, a particular advantage of the present invention is that it is possible to dispense with packaging the implant in a gas-tight and liquid-tight envelope, as described above, if, after the treatment with UV rays according to the invention, the implant is implanted within a reasonable time, for example within one hour. Surprisingly, as was mentioned at the outset, the hydrophilic character of the implant surface treated according to the invention is maintained substantially unchanged during this time, even in contact with atmospheric air. Suitable and relatively compact UV devices are available today and can easily be set up in medical practice. It therefore suffices that an implant which is provided with a roughened but hydrophobic surface, and whose production is described above and known per se, is packaged free of dust. Directly before implantation, the operating surgeon (dental surgeon) can treat the implant, provided with a hydrophobic surface, with the UV rays in the UV device in a manner according to the invention, resulting in a microbe-free (sterilized) implant with a hydroxylated and hydrophilic surface. This hydroxylated and hydrophilic state of the implant surface, and also its biological activity, is thus substantially maintained until implantation.

In this sense, the present invention also relates to a method for implanting an osteophilic implant with a roughened, hydroxylated and hydrophilic surface, said implant being made of titanium or a titanium alloy and being suitable for implantation in bone, characterized in that the implant in the hydroxylated state is treated with high-energy ultraviolet radiation and is implanted in the bone directly after this treatment. The following examples illustrate the invention.

### EXAMPLE 1

A conventional form of a tooth implant in the form of a screw with a diameter of 4 mm and a length of 10 mm was produced. The basic form was obtained by removing material by turning and milling the cylindrical preform in a manner known per se. The surface to be inserted into the bone was then provided with a macro-roughness as described in EP 0 388 576 by sandblasting it with particles of average particle size 0.25-0.5 mm. Subsequently, the roughened surface, that is to say the macro-roughness, was treated with an aqueous hydrochloric acid/sulfuric acid mixture with an HCl:H₂SO₄:H₂O ratio of 2:1:1 at a temperature of 95° C. for five minutes to result in a ratio of the roughened implant surface to the comparable polished surface of 3.6, measured by voltametry in an aqueous electrolyte with 0.15 M NaCl (corresponding to a ratio of 3.9 measured by impedance spectrometry in the 0.1 molar Na₂SO₄ electrolyte). The implant formed in this way was washed thoroughly with pure water to neutral, then dried in atmospheric air at 110° C., and packaged free of dust in a glass ampule.

a) Some of the implants were implanted unchanged (comparative test).

b) The other implants were treated for five minutes in a xenon emitter from the Radium company (Lampenwerk Wipperfurth, Germany) with UV radiation at an emission maximum of 172 nm, at a distance of 0.1 mm in atmospheric air.

The samples according to test a) [without UV radiation] had a wetting angle of over 70° (>70°) and a content of 20-40 atom-% carbon on the surface. The samples according to test b) [after UV radiation] were completely wettable, i.e. they had a wetting angle of 0° (zero degree) and contained 8-13 atom-% carbon on the surface. The low-carbon state of the surfaces of the samples according to test b) remained practically unchanged over a period of at least one day.

The implants according to tests a) and b) were implanted in the upper jaw of a minipig. The anchoring in the bone was measured as the loosening torque of the screw implanted in the upper jaw of the minipig. The results obtained are indicated in Table 1.

TABLE 1

| | Anchoring* | Anchoring* | Anchoring* |
|---|---|---|---|
| | after 2 weeks | after 3 weeks | after 4 weeks |
| | (Ncm) | (Ncm) | (Ncm) |
| Test a) | 20 | 58 | 80 |
| Test b) | 55 | 85 | 110 |

*The anchoring is specified as loosening torque in Ncm (averages).

1. An osteophilic implant with a hydroxylated surface comprising titanium or a titanium alloy, preferably with a rough surface suitable for implantation in bone, wherein the implant is hydrophilic, that is to say substantially free of impurities on the surface.

2. An osteophilic implant with a rough surface comprising titanium or a titanium alloy, produced by a production and preparation process which comprises a surface treatment process active until implantation, comprising the steps of:
a) hydroxylation of the surface of the implant, and
b) treatment of the surface of the implant by ultraviolet radiation for preparing a hydrophilic surface, in particular for destroying and/or removing organic impurities.

3. The implant as claimed in claim 2, wherein the treated implant surface has a content of less than 20 atom-% carbon, in particular of less than 13 atom-%.

4. The implant as claimed in claim 2, wherein the surface is treated with UV radiation in this way in order to achieve hydrophilicity and to free the surface of impurities.

5. The implant as claimed in one of claim 1, wherein said implant is made of a titanium alloy, preferably of a titanium/zircon alloy, which may additionally contain niobium, tantalum or other tissue-compatible metallic additives.

6. The implant as claimed in one of claim 1, wherein said implant constitutes a hip-joint or knee-joint prosthesis or a pin to be screwed into the jaw for attachment of artificial teeth.

7. The implant as claimed in one of claim 1, wherein the implant surface is provided with a macro-roughness, preferably with a screw thread or with depressions in the surface, and if appropriate additionally with a micro-roughness superposed on the macro-roughness.

8. The implant as claimed in claim 7, wherein the macro-roughness lies in the range of 5 μm to 100 μm (in each case peak to valley), in particular in the range of 20 μm to 40 μm, and the superposed micro-roughness lies in the range of 0.5 μm to 20 μm, in particular in the range of 0.5 μm to 10 μm, in particular about 2 μm.

9. The implant as claimed in one of claim 1, wherein the UV rays used are those with a wavelength in the range of 150 nm to 300 nm, preferably in the range of 170 nm to 260 nm, and in particular UV rays with a wavelength of about 184.9 nm and about 253.7 nm.

10. The implant as claimed in one of claim 1, wherein the irradiation takes place for a period of 1 minute to 15 minutes.

11. The implant as claimed in one of claim 1, wherein said implant is packaged in an envelope which is filled with gases inert for the implant surface, preferably with nitrogen, oxygen or noble gas, and is impermeable to gases and liquids.

12. The implant as claimed in claim 11, wherein the envelope is filled partially or completely with pure water, which possibly contains additives.

13. The implant as claimed in claim 12, wherein the water contains alkali metal cations, preferably Na⁺ or K⁺, with corresponding anions in the form of inorganic salts, preferably sodium chloride, potassium chloride, sodium or potassium chlorate, sodium or potassium nitrate, sodium or potassium phosphate, or a mixture of such salts, or divalent cations with the form of water-soluble inorganic salts, preferably Mg⁺², Ca⁺², Sr⁺² and/or Mn⁺², preferably in the form of the chlorides, or water-soluble phosphates and/or phosphonates.

14. The implant as claimed in claim 12, wherein the total amount of cations and anions in each case lies in the range of 50 mEq/l to 250 mEq/l, preferably in the range of 100 mEq/l to 200 mEq/l, and preferably about 150 mEq/l.

15. The implant as claimed in one of claim 11, wherein the gas-tight and liquid-tight envelope is an ampule made of glass, metal, a synthetic polymer or another gas-tight and liquid-tight material, or consists of a combination of these materials.

16. The osteophilic implant as claimed in claim 1, with a roughened hydroxylated and hydrophilic surface, said implant being made of titanium or a titanium alloy, wherein the implant, in the hydroxylated state, was treated with high-energy ultraviolet radiation.

17. A process for implanting an osteophilic implant with a hydroxylated and hydrophilic surface, preferably with a rough surface suitable for implantation in bone, said implant being made of titanium or a titanium alloy, wherein the implant, in the hydroxylated state, is treated with ultraviolet radiation, the UV rays used being those with a wavelength in the range of 150 nm to 300 nm, preferably in the range of 170 nm to 260 nm, and in particular UV rays with a wavelength of about 184.9 nm and about 253.7 nm, and the implant is implanted in the bone directly after this treatment.

18. A process for implanting an osteophilic implant with a hydroxylated and hydrophilic surface, preferably with a rough surface suitable for implantation in bone, said implant being made of titanium or a titanium alloy, wherein the implant, in the hydroxylated state, is treated with ultraviolet radiation, the UV rays used being those with a wavelength in the range of 150 nm to 300 nm, preferably in the range of 170 nm to 260 nm, and in particular UV rays with a wavelength of about 184.9 nm and about 253.7 nm, and the hydrophilic state of the surface achieved in this way is preserved by suitable storage, with the implantation in the bone taking place at a later time.

19. Use of an osteophilic implant made of titanium or a titanium alloy, preferably with a rough surface suitable for implantation, the surface being hydroxylated and being treated by UV radiation, in particular high-energy irradiation, in such a way that organic impurities on the surface are destroyed or rendered inactive, for the purpose of the implantation of a dental implant.
